(19) United States
(12) Patent Application Publication
(10) Pub. No.: US 2008/0247989 A1
(43) Pub. Date: Oct. 9, 2008

(54) Reishi - Mediated Enhancement of Human Tissue Progenitor Cell Adhesion and Differentiation

(76) Inventors: Daniel Tzu-Bi Shih, Taipei (TW); Wan-Yu Chen, Taipei (TW); Chi-Huey Wong, Rancho Santa Fe, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 11/859,738

(22) Filed: Sep. 21, 2007

Related U.S. Application Data
(60) Provisional application No. 60/846,676, filed on Sep. 21, 2006.

(51) Int. Cl. A61K 38/20; A61K 36/074

(52) U.S. Cl. 424/85.2; 424/195.15; 424/93.7

(57) ABSTRACT

The present disclosure provides medicinally active extracts and fractions, and methods for using the same to increase eukaryotic cell adhesion, to increase differentiation of eukaryotic cells to produce increased numbers of B cells dendritic cells and chodrocytes, and to maintain undifferentiated hematopoietic cells. These methods and useful for modulating immune response, modulating hematopoietic activity, and engineering certain types of eukaryotic tissues.

### RELATED APPLICATION

This disclosure claims the benefit of U.S. Provisional Application No. 60/846,676, filed Sep. 21, 2006, the disclosure of which is incorporated herein by reference in its entirety.

### BACKGROUND

The present disclosure provides medicinally active extracts and fractions, and methods of preparing the same, from components of Ganoderma lucidum and Ganoderma tsugae, otherwise known as “Reishi”. These extracts and fractions have been found to be especially active in modulating immune response and in increasing hematopoietic activity.

Cell adhesion molecules play a key role in mobilizing cell-cell interactions, particularly during embryonic development, neural plasticity, and tumor metastasis. Glycoside conjugates on cellular membranes play an important role in determination of stem cell and stem cell-derived eukaryotic cell homing, mobilization, differentiation, morphogenesis, and adhesion. Thus, identifying and characterizing glycoconjugate and membrane receptor expression on normal tissue progenitor cells is critical in controlling the health, disease state, and homeostasis of eukaryotic cells and eukaryotic cell-based organisms.

### SUMMARY

In one implementation, a method is disclosed comprising administering purified reishi extract to a subject, wherein mononuclear cell expression of cell surface markers is increased by at least 1%.

In another implementation, the cell surface markers are VCAM and/or NCAM.

In another implementation, the cell surface markers are selected from the group consisting of immature dendritic cell markers.

In another implementation, the immature dendritic cell markers are selected from the group consisting of CD1a, CD14, CD40, CD80, and CD86.

In another implementation, the cell surface markers are selected from the group consisting of mature dendritic cell markers.

In another implementation, the mature dendritic cell markers are selected from the group consisting of CD83.

In another implementation, the cell surface markers are selected from the group consisting of hematopoietic cell markers.

In another implementation, the hematopoeitic cell markers are selected from the group consisting of CD34, CD38, CD133, and CXCR4.

In another implementation, the cell surface markers are selected from the group consisting of B cell markers.

In another implementation, the B cell markers are selected from the group consisting of CD19.

In another implementation, purified reishi is co-administered to a subject with at least one cytokine selected from the group consisting of IL-4 and GM-CSF.

In another implementation a method is disclosed comprising administering purified reishi extract to a subject, wherein MSC and/or PLA expression of cell surface markers is increased by at least 1%.

In another implementation, the cell surface markers are selected from the group consisting of BMP-2, aggrecan, and IL-1.

In another implementation, reishi is co-administered to a subject with at least one compound selected from the group consisting of insulin, TGF-B1, and/or ascorbate-2-phosphate.

In another implementation, purified reishi is administered to a subject, wherein the percentage of subject MSC and/or PSA cells that lose expression of the CD34+/CD38− cell proteome is decreased by at least 1%.

In another implementation, a combination is disclosed comprising an amount of skeleton forming agent; and an amount of reishi extract.

In another implementation, the combination is further comprised of an amount of mononuclear cells.

In another implementation, the combination is further comprised of an amount of MSC and/or PLA cells.

In another implementation, the combination is further comprised of a medical device.

In another implementation, the combination is implantable into an organism.

### DRAWINGS

FIG. 1 depicts the percentage of suspended and adherent mononuclear cells expressing particular cell surface markers in vitro when incubated with and without purified reishi, as characterized by FACS analysis.

FIGS. 2A-2D qualitatively depict the amount of adherence of mononuclear cells markers in vitro when incubated with and without purified reishi, as characterized by visual observation of the relative confluence of the cells on the incubation vessel.

FIG. 2E qualitatively depicts the morphological changes in mononuclear cells in vitro when incubated with and without purified reishi, as characterized by visual observation of the morphology of the incubated cells.

FIG. 3A depicts the relative level of expression of V-CAM and N-CAM cell surface markers expressed by cells in vitro, assayed by Western blot analysis.

The axes on FIG. 3A are as follows: Horizontal (left to right): CD1a(+)/CD14(−); CD40; CD80; CD83; CD86; Vertical: CD marker expression %.

FIGS. 3B and 3C depict the percentage of cells expressing particular cell surface markers in vitro when incubated with and without purified reishi, as characterized by FACS analysis.

The axes on FIG. 3B are as follows: Horizontal (left to right): CD1a(+)/CD14(−); CD40; CD80; CD83; CD86; Vertical: CD marker expression %.

FIG. 3D depicts the relative population of cells expressing the CD19+ cell surface marker in vitro when incubated with and without purified reishi, as characterized by FACS analysis.

The axes on FIG. 3D are as follows: Horizontal (left to right): CD1a; CD3; CD14; CD16; CD19; CD34+CD45; CD56; CD83; Vertical: ratio to contr. 0; 0.5; 1; 1.5; 2; 2.5; 3; 3.5.

FIG. 3E depicts the relative population of cells expressing the CD83 cell surface market in vitro when incubated with and without purified reishi, as characterized by FACS analysis.

The axes on FIG. 3E are as follows: Horizontal: CD1a; CD3; CD14; CD19; CD56; CD83; Vertical: ratio to contr. 0; 0.5; 1; 1.5; 2; 2.5; 3; 3.5.

FIGS. 3F and 3G depict the relative levels of various cytokines expressed by cells incubated in vitro with and without reishi extract, as characterized by mRNA (cDNA, PCR) analysis.

The axes on FIG. 3F are as follows: Horizontal: IL-1; IL-6; MCP-1; RANTES; GRO; GRO-1; MIP-1; Vertical: 0; 20; 40; 60; 80; 100. The axes on FIG. 3G are as follows: Horizontal: angiogenin; IL-4; PARC; Vertical: 0; 20; 40; 60; 80; 100.

FIGS. 4A and 4B depict the relative number of PSA and MSC cells incubated in vitro with and without reishi extract that express the CD34+ cell surface marker and the CD34+/CD38− proteome, assessed by FACS and/or total mRNA analysis.

The axes on FIG. 4A are as follows: Vertical: 0; 5; 10; 15; 20; 25; Horizontal: DAY1; DAY4; DAY7; DAY12. The axes on FIG. 4B are as follows: Vertical: 0%; 20%; 40%; 60%; 80%; 100%; Horizontal: CD34+; CD34+/CD38−; CD34+/CD38+; 38+; 38−.

FIGS. 4C-4F depict the relative number of cells incubated in vitro with and without various reishi extract fractions that express the CD34+ cell surface marker and the CD34+/CD38− proteome, assessed by FACS and/or total mRNA analysis.

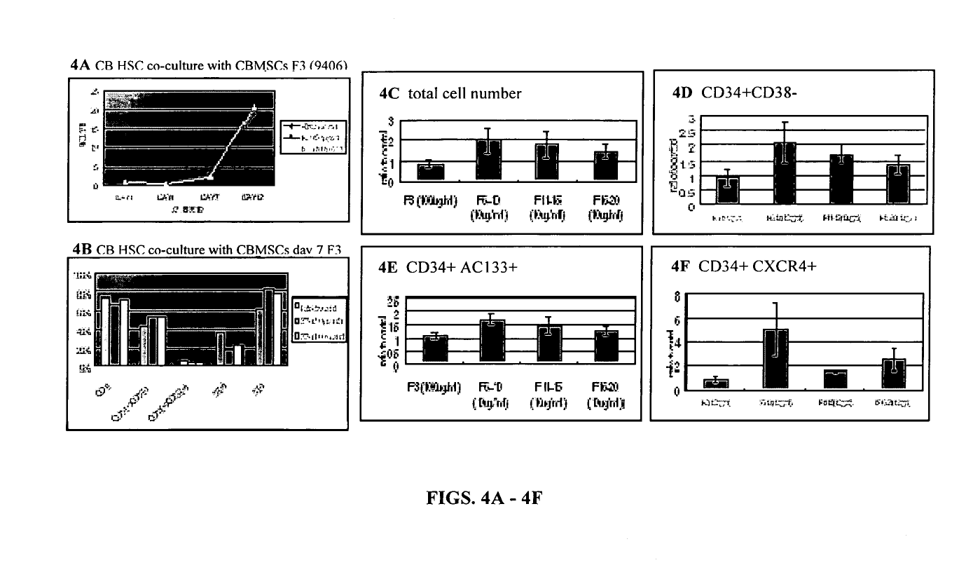

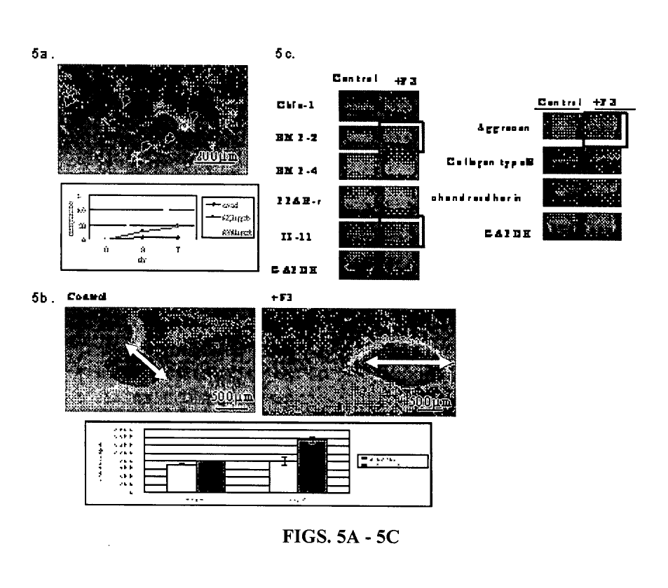

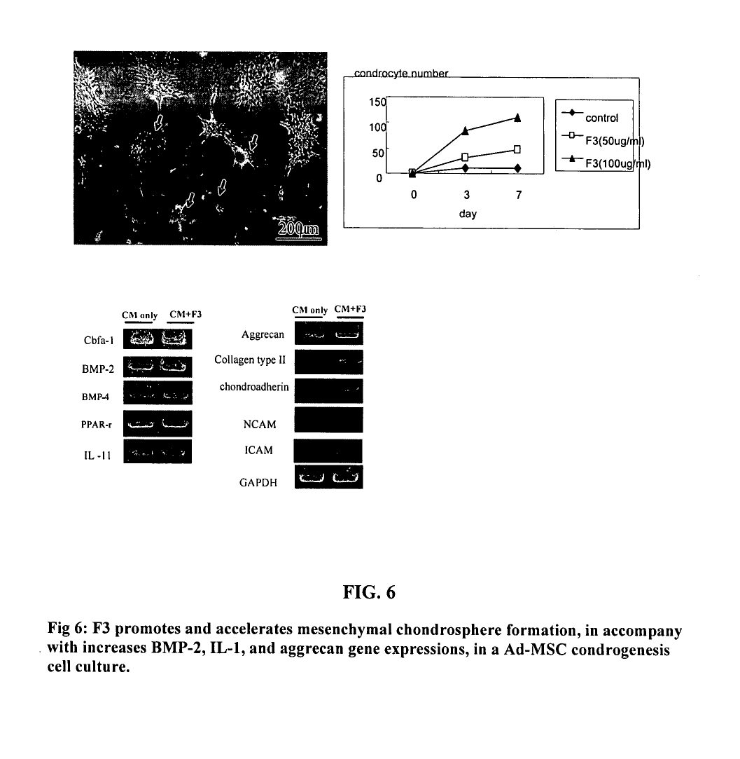

The axes on FIG. 4C are as follows: Vertical: “ratio to control” 0, 1, 2, 3; Horizontal: F3(100 ug/ml); F6-10 (10 ug/ml); F11-15 (10 ug/ml); F16-20 (10 ug/ml). The axes on FIG. 4D are as follows: Vertical: “ratio to control” 0, 0.5, 1, 1.5, 2, 2.5, 3; Horizontal: F3(100 ug/ml); F6-10 (10 ug/ml); F11-15 (10 ug/ml); F16-20 (10 ug/ml). The axes on FIG. 4E are as follows: Vertical: “ratio to control” 0, 0.5, 1, 1.5, 2, 2.5, 3; Horizontal: F3(100 ug/ml); F6-10 (10 ug/ml); F11-15 (10 ug/ml); F16-20 (10 ug/ml). The axes on FIG. 4F are as follows: Vertical: “ratio to control” 0, 2, 4, 6, 8; Horizontal: F3(100 ug/ml); F6-10 (10 ug/ml); F11-15 (10 ug/ml); F16-20 (10 ug/ml).

FIGS. 5A-5C and FIG. 6 depict the relative number of mesenchymal stem cells incubated with and without reishi extract in vitro that differentiate to chondrocyte morphology, characterized by chondrosphere formation (via phase-contrast microscopy), Western blot analysis of various cell proteome components and cell surface markers and morphological and cell count analyses of chondrocyte differentiation.

The axes on 5A are as follows: Vertical: “condrocyte (SIC) number” 0, 50, 100, 150; Horizontal: “day” 0, 3, 7. The Legends are: control, F3 (50 ug/ml), F3(100 ug/ml).

The axes on 5B are as follows: Vertical: “Chondrocyte” 0; 200; 400; 600; 800; 1000; 1200; 1400; 1600; Horizontal: day 3; day 7. The Legends are: control, +F3 (100 ug/ml).

In FIG. 6: In left hand Western blot: Vertical: Chfa-1; BMP-2; BMP-4; PPAR-r; IL-11; Horizontal: CM only; CM+F3. In right hand Western blot: Vertical: Aggregan; Collagen type II; chondroadherin; NCAM; ICAM; GAPDH; Horizontal: CM only; CM+F3. In graph: Vertical column: “condrocyte (SIC) number” 0; 50; 100; 150; Horizontal column: “day” 0; 3; 7. Legend: -control, −F3 (50 ug/ml), −F3 (100 ug/ml).

Table 1 depicts the carbohydrate composition of crude Reishi extract, assayed via the TMS method.

Table 2 depicts the amino acid composition of crude Reishi extract, assayed via a well-established method.

Table 3 depicts the relative carbohydrate compositions of various purified Reishi fractions, assayed via the TMS method

Table 4 depicts the cytokine expression of mouse splenocytes treated with various Reishi samples, assayed via analysis of total RNA (e.g. RT-pCR).

Table 5 depicts various primer sequences used in RT-PCR, used to assay levels of expression of various cytokines of interest.

### DETAILED DESCRIPTION

For the purposes of describing the present disclosure, the following terms are intended to refer to the associated definitions as described below:

“Angiogenesis” means a physiological process involving the growth of new blood vessels from pre-existing vessels

“Adherent Cells” means cells that remain attached to the sides of an incubation vessel subsequent to aspiration of incubation media. Adherent cells may be subsequently dissociated from the sides of an incubation vessel by means of chemical and/or physical processes, e.g. through application of a trypsin solution.

“Administering” means oral, or parenteral administration including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection and infusion as well as co-administration as a component of any medical device or object to be inserted (temporarily or permanently) into the body.

“Aggrecan” means a proteoglycan, or a protein modified with carbohydrates. Along with collagen, aggrecan forms a major structural component of cartilage, particularly articular cartilage.

“Amount of cells adhering” means an assay of the relative number of cells adhering to the surfaces of an incubation vessel. This assay may be carried out in a variety of ways, such as through visual observation of the degree of confluence of adherent cells on the surfaces of an incubation vessel, or through trypsinization of the adherent cells and subsequent counting of a representative sample of the trypsinized cells in solution.

“B cell” means any of a class of lymphocytes that play a large role in the humoral immune response as opposed to the cell-mediated immune response that is governed by T cells. B cells are produced in the bone marrow of most mammals and are therefore called B cells. The principal function of B cells is to make antibodies against soluble antigens. B cells are an essential component of the adaptive immune system.

“BMP-2” means bone morphogenic protein, a type of cytokine.

“Buffer Solution” means a solution which resists change in hydrogen ion and hydroxide ion concentration (and consequently pH) upon addition of small amounts of acid or base, or upon dilution. Buffer solutions consist of a weak acid and its conjugate base (more common) or a weak base and its conjugate acid (less common).

“Cartilage” means a tough, elastic, fibrous connective tissue found in various parts of the body, such as the joints, outer ear, and larynx. A major constituent of the embryonic and young vertebrate skeleton, it is converted largely to bone with maturation.

“CD” means cluster of differentiation, a protocol used for the identification and investigation of cell surface molecules present on leukocytes. CD molecules can act in numerous ways, often acting as receptors or ligands (the molecule that activates a receptor) important to the cell. A signal cascade is usually initiated, altering the behavior of the cell (see cell signaling). Some CD proteins do not play a role in cell signalling, but have other functions, such as cell adhesion.

“Cell Surface Markers” means a protein that is present in the cell surface of a eukaryotic cell, as well as any gene expression product specific to that particular protein (whether characterized in vivo or in vitro)(for example, mRNA or cDNA).

“Centrifuge” means an apparatus consisting essentially of a compartment spun about a central axis to separate contained materials of different specific gravities, or to separate colloidal particles suspended in a liquid.

“Chondrocyte” means a type of eukaryotic cell found in cartilage. Chondrocytes produce and maintain the cartilaginous matrix, which consists mainly of collagen and proteoglycans. The progenitors of chondrocytes are mesenchymal stem cells.

“Chondrogenesis” means the process by which cartilage is formed.

“Cytokine” means any of a group of proteins and peptides that are used in organisms as signaling compounds. These chemical signals are similar to hormones and neurotransmitters and are used to allow one cell to communicate with another.

“Dendritic Cells” means immune cells that form part of the mammalian immune system. Their main function is to process antigen material and present it on the surface to other cells of the immune system, thus functioning as antigen-presenting cells.

“Dendritic Cell Markers” means any of a group of cell surface molecules found generally on the surface of dendritic cells.

“Differentiate” means the process by which eukaryotic cells acquire a “type” (e.g. dendritic cell, chondrocyte); e.g. a change in cellular morphology without a requirement of a change in genetic material.

“Endothelial” means the thin layer of cells that line the interior surface of blood vessels, forming an interface between circulating blood in the lumen and the rest of the vessel wall.

“Expression” means the process by which inheritable information which comprises a gene, such as the DNA sequence, is made manifest as a physical and biologically functional gene product, such as protein or RNA. Expresson may be quantitated by immunological (e.g. MACS, FACS) and/or by molecular biology (e.g. total RNA analysis) techniques.

“FACS” means Fluorescence Activated Cell Sorting (e.g. flow cytometry). FACS is a powerful method used to study and purify cells. Individual cells held in a thin stream of fluid are passed through one or more laser beams cause light to scatter and fluorescent dyes to emit light at various frequencies. Photomultiplier tubes (PMT) convert light to electrical signals and cell data is collected. Cell sub-populations are identified and sorted at high purity (˜100%) based upon the charge of a flourescent dye linked to a particular cell type via an antibody-antigen relationship.

“Ficoll-Hypaque” means a density-gradient centrifugation technique for separating lymphocytes from other formed elements in the blood; the sample is layered onto a Ficoll-sodium metrizoate gradient of specific density; following centrifugation, lymphocytes are collected from the plasma-Ficoll interface.

“Gel filtration” means separation of proteins, peptides, and oligonucleotides on the basis of size. Molecules move through a bed of porous beads, diffusing into the beads to greater or lesser degrees. Smaller molecules diffuse further into the pores of the beads and therefore move through the bed more slowly, while larger molecules enter less or not at all and thus move through the bed more quickly. Both molecular weight and three dimensional shape contribute to the degree of retention. Gel Filtration Chromatography may be used for analysis of molecular size, for separations of components in a mixture, or for salt removal or buffer exchange from a preparation of marcromolecules.

“Glycoconjugate” means a type of compound consisting of carbohydrate units covalently linked with other types of chemical constituent.

“Glycoprotein” means proteins that contain oligosaccharide chains (glycans) covalently attached to their polypeptide backbones.

“Glycoside” means certain molecules in which a sugar part of the molecule is bound to some other part of the molecule.

“Glycosylation” means the process or result of addition of saccharides to proteins and lipids.

“GM-CSF” means Granulocyte-macrophage colony-stimulating factor, a cytokine.

“Hematopoietic Cells” means blood forming stem cells. T cells and B cells, among other cell types, arise from these cells.

“Hematopoietic Lineage Markers” means any of a group of cell surface molecules found generally on the surface of hematopoietic stem cells.

“Immature Dendritic Cells” means dendritic cells characterized by high endocytic activity and low T-cell activation potential. Immature dendritic cells are capable of phagocytosing pathogens and other sources of protein.

“Increased Expression of Cell Markers” means an increased quantity of a particular type of cell surface protein or mRNA molecule coding for that particular type of cell surface protein. Various protein molecules are associated with particular eukaryotic cell morphologies. Increased expression may be assayed using antibody—linked cell sorting (“FACS”) or through magnetic-activated cell sorting (“MACS”). Alternately, increased expression may be assayed by use of RT-PCR, in which the quantity of intracellular expression of mRNA coding for production of a particular type of CD molecule is indirectly determined.

“Incubate” means to grow and/or maintain eukaryotic cells in vitro in a vessel (optionally, plastic or glass) and in a liquid medium at conditions of approximately 37° Celsius, 5% carbon dioxide and some degree of humidity. The incubated eukaryotic cells are optionally supplemented with any combination of growth media, cytokines and/or other buffering or other solutions.

“Interleukin” means any of a group of cytokines (secreted signaling molecules) that were first seen to be expressed by white blood cells (leukocytes, hence the -leukin) as a means of communication (inter-). The function of the immune system depends in a large part on interleukins, and rare deficiencies of a number of them have been described, all featuring autoimmune diseases or immune deficiency. Interleukins are commonly designated using an abbreviation: e.g. IL-1, IL-2, etc.

“Lose Expression” means the process by which a particular expressed gene (manifest, e.g. in a particular protein or mRNA sequence) is no longer present or is expressed in reduced quantities or frequencies in a eukaryotic cell. Loss of expression is quantifiable via MACS or FACS analysis or via total mRNA analysis.

“Lyophilize” means a freeze-drying dehydration process typically used to preserve a perishable material or make the material more convenient for transport. Freeze drying works by freezing the material and then reducing the surrounding pressure and adding enough heat to allow the frozen water in the material to sublime directly from the solid phase to gas.

“Macrophage” means cells within the tissues that originate from specific white blood cells called monocytes. Monocytes and macrophages are phagocytes, acting in both nonspecific defense (or innate immunity) as well as specific defense (or cell-mediated immunity) of vertebrate animals. Their role is to phagocytose (engulf and then digest) cellular debris and pathogens either as stationary or mobile cells, and to stimulate lymphocytes and other immune cells to respond to the pathogen.

“MACS” means Magnetically Activated Cell Sorting. MACS is a separation technique that isolates rare cells from whole blood by binding the cells to anti-body labeled paramagnetic beads. The blood is passed through a column with a high magnetic field gradient that traps the paramagnetic beads.

“Mature Dendritic Cells” means dendritic cells that have come into contact with a pathogen and are capable of presenting pathogen protein fragments at their cell surfaces.

“Mesenchyme” (also known as embryonic connective tissue) means the mass of tissue that develops mainly from the mesoderm (the middle layer of the trilaminar germ disc) of an embryo. Viscous in consistency, mesenchyme contains collagen bundles and fibroblasts. Mesenchyme later differentiates into blood vessels, blood-related organs, and connective tissues.

“Mononuclear cells” means large, phagocytic mononuclear leukocytes produced in the vertebrate bone marrow and released into the blood and tissues where they develop into macrophages; contain a large, oval or somewhat indented nucleus and surrounded by voluminous cytoplasm and numerous organelles. For purposes of describing the present disclosure, mononuclear cells includes mononuclear cells in vitro, in vivo and in vivo but subsequently isolated and extracted.

“Morphology” means the outward appearance (shape, structure, color, pattern) and/or identity of a eucaryotic cell, organism, or organism component.

“mRNA” means Messenger Ribonucleic Acid. mRNA is a molecule of RNA encoding a chemical “blueprint” for a protein product.

“MSC” means a type of bone marrow-derived stem cell.

“NCAM” means a cell surface molecule important in mediating binding of eukaryotic cells with other eukaryotic cells and with various extracellular matrix components.

“PBMNC” means a peripheral blood—derived mononuclear cell.

“PBS” means Phosphate—Buffered Saline, a buffer.

“PLA” means an adipose (fat tissue)—derived stem cell

“Percentage of cells demonstrating differentiated chondrocyte morphology” means an assessment of the number of hematopoietic or otherwise differentiatable eukaryotic cells demonstrating differentiated chondrocyte morphology. This change in morphology is quantifiable is through observance via phase-contrast microscopy; measurement of increased N-CAM presence on cell surface (e.g. via mRNA or FACS analysis); and measurement of corresponding increase in intracellular BMP-2, IL-1 and/or aggrecan gene expression (e.g. via mRNA analysis).

“Percentage of cells expressing CD34+, CXCR4+ and/or CD38−” means an assay of the number of eukaryotic cells expressing CD34+, CXCR4+ and/or CD38− is quantifiable through measurement of CD34+, CXCR+ and/or CD38− expression (e.g. via mRNA, FACS and/or MACS analysis).

“Pericyte” means a mesenchymal-like cell, associated with the walls of small blood vessels. As relatively undifferentiated cells, pericytes serve to support these vessels, but it can differentiate into a fibroblast, smooth muscle cell, or macrophage as well if required.

“Proteome” means the complete set of expressed proteins present in a eukaryotic cell at a given time, assayable via mRNA, MACS and FACS analysis.

“Purified Reishi” means a reishi extract prepared as described in U.S. Nonprovisional application Ser. No. 11/553,402, incorporated by reference herein, wherein the purified reishi is comprised of a polysaccharide or glycopeptide containing terminal fucose residues.

“Reishi” means the name for one form of the mushroom Ganoderma lucidum, and its close relative Ganoderma tsugae.

“Stem cell” means an unspecialized cell that gives rise to a specific specialized cell, such as a blood cell.

“Stromal Cells” means connective tissue cells of an organ found in the loose connective tissue. These are most often associated with the uterine mucosa (endometrium), prostate, bone marrow precursor cells, and the ovary as well as the hematopoietic system and elsewhere. These are the cells which make up the support structure of biological tissues and support the parenchymal cells.

“Subject” means, but is not limited to, any eukaryotic cell or eukaryotic cell-based organism, whether administered in vivo or in vitro to that cell or cell-based organism.

“Suspension Cells” means incubated eukaryotic cells that have not adhered to the sides of the incubation vessel.

“0.1 N Tris buffer” means a buffer solution.

“VCAM” means a cell surface molecule (otherwise known as CD-106). VCAM-1 promotes the adhesion of lymphocytes, monocytes, eosinophils, and basophils.

Detailed implementations of the present disclosure are disclosed herein; however, it is to be understood that the disclosed implementations are merely illustrative of the implementation that may be embodied in various forms. In addition, each of the examples given in connection with the various implementations of the disclosure are intended to be illustrative, and not restrictive. Further, the figures are not necessarily to scale, and some figures may be exaggerated to show details of particular components. Therefore, specific structural and functional details disclosed herein are not to be interpreted as limiting, but merely as a representative basis for teaching one skilled in the art to variously employ the present disclosure.

In one implementation, a method is disclosed comprising homogenizing reishi tissue; dissolving the reishi extract in water; stirring the reishi extract/water mixture for at least about 24 hours while maintaining the temperature of the reishi extract/water mixture at a temperature of at least about 40 Celsius; centrifuging the reishi extract/water mixture for a sufficient amount of time to remove insoluble materials; evaporating water from the centrifuged reishi extract/water mixture at a temperature of at least about 350 Celsius in order to remove at least a portion of the water from the reishi extract/water mixture; lyophilizing the resultant concentrated reishi extract/water mixture; resuspending the lyophilized reishi extract in a liquid phase; and purifying the resuspended reishi extract.

In another implementation, the lyophilized reishi extract is resuspended in 0.1 N Tris buffer. In another implementation, the lyophilized reishi extract is resuspended and the resultant liquid suspension is adjucted to a pH of 7.0. In another implementation, the resuspended reishi extract is purified by use of gel filtration. In another implementation, the resuspended reishi extract gel filtration is collected as a series of fractions, each of which is subjected to anthrone analysis or the phenol-sulfuric acid method in order to detect sugar components. In another implementation, the filtered resuspended reishi extract is dialyzed to remove excessive salt. In another implementation, the reishi extract is subjected to anion exchange, eluted with sodium chloride solution and optionally re-fractionated. In another implementation, the filtered resuspended reishi extract is re-lyophilized subsequent to filtration.

In another implementation, a method is disclosed comprising combining a plurality of mononuclear cells with purified reishi extract; and incubating the mononuclear cells and reishi extract for a sufficient amount of time so as to increase the mononuclear cell expression of cell markers.

In another implementation, the combination further includes conditional culture medium. In another implementation, the conditional culture medium is AIM-V. In another implementation, the AIM-V conditional medium is serum-free. In another medium, the AIM-V conditional culture medium is supplemented with fetal bovine serum. In another implementation, the AIM-V conditional culture medium is supplemented with IL-4 and GM-CSF.

In another implementation, the cell surface markers are VCAM and/or NCAM.

In another implementation, the cell surface markers are selected from the group consisting of immature dendritic cell markers. In another implementation, the immature dendritic cell markers are selected from the group consisting of CD1a, CD14, CD40, CD80, and CD86.

In another implementation, the cell surface markers are selected from the group consisting of mature dendritic cell markers. In another implementation, the mature dendritic cell markers are selected from the group consisting of CD83.

In another implementation, the cell surface markers are selected from the group consisting of hematopoietic cell markers. In another implementation, the hematopoeitic cell markers are selected from the group consisting of CD34, CD38, CD133, and CXCR4.

In another implementation, the cell surface markers are selected from the group consisting of B cell markers. In another implementation, the B cell markers are selected from the group consisting of CD19.

In another implementation, the combination of purified reishi extract and mononuclear cells further comprises at least one cytokine selected from the group consisting of IL-4 and GM-CSF.

In another implementation, the purified reishi extract (alone or in combination with mononuclear cells) is administrable to a person in need of treatment.

In another implementation, a method is disclosed comprised of administering a sufficient amount of purified reishi extract subject to increase expression of cell surface markers selected from the group consisting of BMP-2, aggrecan, and IL-1.

In another implementation, the combination further includes conditional culture medium. In another implementation, the conditional culture medium is AIM-V. In another implementation, the AIM-V conditional medium is serum-free. In another medium, the AIM-V conditional culture medium is supplemented with fetal bovine serum. In another implementation, the AIM-V conditional culture medium is supplemented with IL-4 and GM-CSF.

In another implementation, purified reishi extract is provided as part of a kit option TGF, chondrotin, and/or glucosamin skeleton forming agents. In another implementation, a combination is disclosed comprising co-administering the combination reagents with one or more skeleton forming agents with or without a medical device within which the reagents and/or skeleton forming agents are impregnated to a subject.

In another implementation, the combination of the MSC and/or PLA cells and purified reishi extract is further comprised of compounds selected from the group consisting of insulin, TGF-B1, and/or ascorbate-2-phosphate.

In another implementation, a method is disclosed comprising the steps of combining a plurality of MSC and/or PLA cells in vitro with purified reishi extract; incubating the MSC and/or PLA cells and reishi extract for an amount of time; and quantitatively assessing the percentage of incubated MSC and/or PSA cells that retain CD34+, CXCR4+ and/or CD38− as cell proteome characteristics wherein the percentage of cells expressing CD34+, CXCR4+ and/or CD38− as a percentage of the total number of cells incubated with reishi extract is increased as compared to the percentage of cells expressing CD34+, CXCR4+ and/or CD38− as a percentage of an equivalent total number of cells incubated without purified Reishi extract.

In another implementation, the purified reishi extract (alone or in combination with MSC and/or PSA cells) is administrable to a person in need of treatment.

Materials. Crude Reishi extract (prepared via alkaline extraction (0.1 N NaOH), neutralization and ethanol precipitation) was obtained from Pharmanex Co. (CA, USA). Immobiline DryStrip (pH 3-10 NL (non-linear), 18 cm) and IPG buffer (pH 3-10 NL) were purchased from Amersham Pharmacia Biotech (Uppsala, Sweden). CHAPS, Tris buffer, agarose, iodoacetamide and alpha-cyano-4-hydroxycinnamic acid were from Sigma Co. (St. Louis, Mo., USA); dithioerythreitol (DTE) was from Merck Co. (Darmstast, Germany); acrylamide, ammonium persulfate (APS) and TEMED were from Bio-Rad (Hercules, Calif., USA); sodium dodecyl sulfate (SDS) and glycine were from Fluka (Buchs, Switzerland); sequencing grade trypsin was from Promega (Madison, Wis., USA).

Purification of Reishi extract. Twenty eight mg of the crude extract were dissolved in 2 mL of Tris buffer (pH 7.0, 0.1 N) and centrifuged to remove the insoluble materials (7 mg). The supernatant was purified by gel filtration chromatography using a Sephacryl S-500 column (100 cm×1.6 cm) with 0.1 N Tris buffer (pH 7.0) as the eluent. The flow rate was set at 0.5 mL/min, and 7.5 mL per tube was collected. After the chromatography, each fraction was subjected to anthrone analysis to detect sugar components. Five fractions were collected (fractions 1-5), each dialyzed to remove excessive salt and lyophilized to give 1.0, 6.2, 5.3, 2.1, and less than 1 mg, respectively.

Anthrone colorimetric method.⁶ Each 1.5 mL of anthrone (9,10-dihydro-9-oxoanthracene) solution (0.2 g anthrone dissolved in 100 mL of conc. sulfuric acid) in a series of test tubes immersed in an ice water bath was carefully overlayed with 1.5 mL of sample (20-40 μg/mL of D-glucose or equivalent). After all additions had been made, the tubes were shaken rapidly and then replaced in an ice water bath. The tubes were heated for 5 min a boiling water bath and then cooled; the optical densities were read within an hour at 625 nm against distilled water. Standards, reagent blanks and unknowns were run in triplicate because of likely contamination by other carbohydrate sources. Calculations were made on the basis that the optical densities are directly proportional to the carbohydrate concentration.

Mitogen-induced proliferation of spleen cells and colorimetric MTT assay.⁷ Whole spleen cells were harvested from BALB/c male mice (six weeks old), suspended in RPMI-1640 medium containing 10% FCS (fetal calf serum), and centrifuged to remove the supernatant. The collected precipitated cells were first suspended in 1 mL of RBC lysis buffer (8% NH₄Cl), then 14 mL more of the same lysis buffer were added to destroy red blood cells. After 1 min, the solution was diluted with 15 mL RPMI-1640 medium to stop the reaction, centrifuged to collect the cells, and adjusted the cell final concentration to 2×10⁶ cells/mL with RPMI-1640 medium. Concanavalin A (Con A, final conc: 1 μg/mL) was added to the resulting mixture. The cells were incubated with or without a Reishi extract (or partially purified fraction) in 96-well ELISA plates at 37° C. with 5% CO₂ for 72 h. The cell proliferation was measured based on the MTT assay.

MTT (3-(4,5-dimethylthiazol-2-yl)-2,5-diphenyl tetrazolium bromide) was dissolved in phosphate buffered saline (PBS) at 5 mg/mL and filtered to sterilize and remove a small amount of insoluble residue present in some batches of MTT. At the times indicated below, 25 μL of MTT solution was added to all wells of an assay, and plates were incubated at 37° C. for 4 h. Acid-isopropanol (100 μL of 0.04 N HCl in isopropanol) was added to all wells and mixed thoroughly to dissolve the dark blue crystals. After a few minutes at room temperature to ensure that all crystals were dissolved, the plates were read on a Perkin Elmer ELISA reader (HTS 7000 plus), using a test wavelength of 570 nm, a reference wavelength of 620 nm. Plates were normally read within 1 h after the addition of isopropanol.

Reverse transcription (RT) and polymerase chain reaction (PCR).⁸ Mouse spleen cells were aseptically removed from healthy mice (BALB/c male mice, six weeks old), adjusted to an ideal cell concentration (4×10⁶ cells/mL) and incubated in RPMI-1640 medium containing 10% of FCS (fetal calf serum) at 37° C. with 5% CO₂. After 6 h, the cells were subjected to RNA extraction using Qiagen RNAeasy mini kit to obtain ˜1 μg of the desired RNA. Reverse transcription (RT) was performed using the Thermoscript R/T PCR System, and the Themoscript system protocol 1, from Gibco BRL. The reaction was carried out as follows: 8 μL of RNA, 2 μL of primer (Oligo(dT)₂₀) (SEQ ID NO:23), 2 μL of 10 mM dNTP Mix, and DEPC H₂O (0.1% diethylpyrocarbonate-treated H₂O) was added to each tube, which was then incubated at 65° C. for 5 min and immediately put on ice. The following was added to each tube as a 8 μL mixture: 4 μL of 5×cDNA buffer, 1 μL of 0.1 M dithiothreitol (DTT), 1 μL of RNaseOut (a ribonuclease inhibitor) and 1 μL of Thermoscript R/T, and 1 μL of DEPC water. The mixture was incubated at room temperature for 10 min and then 55° C. for 30 min to allow first strand of cDNA synthesis. Enzyme activity was terminated by incubating the reactions at 85° C. for 5 min and the tubes were then placed on ice for 10 min. The samples were stored at −20° C. until used for PCR.

Each sample (3 μL) was added to each reaction tube and the following reagents were added as a 47 μL mix: 5 μL of 10×PCR buffer, 4 μL of 10 mM dNTP Mix, 2 μL of each primer (10 OD/mL, sense and anti-sense), 33 μL of DEPC H₂O, and 1 μL of ProZyme® (DNA polymerase, from PROtech Technology). The reaction tubes were placed in a Strategene PCR Robocycler (Gradient 96) and run under the following condition: 1 cycle at 92° C. for 2 min (initial denaturation), then 30 consecutive cycles of 91° C. for 10 s (denaturation), 59° C. for 25 s (primer annealing) and 72° C. for 25 s (primer extension). The reactions were analyzed by gel electrophoresis.

Sugar composition analysis—TMS method. For monosaccharide analysis, the polysaccharide extracts/fractions were methanolyzed with 0.5 M methanolic-HCl (Supelco) at 80° C. for 16 h, re-N-acetylated with 500 μL of methanol, 10 μL of pyridine and 50 μL of acetic anhydride, and then treated with the Sylon HTP® trimethylsilylating reagent (Supelco) for 20 min at room temperature, dried and redissolved in hexane. GC-MS analysis of the trimethylsilylated derivatives was carried out using a Hewlett-Packard (HP) Gas Chromatograph 6890 connected to a HP 5973 Mass Selective Detector. Samples were dissolved in hexane prior to splitless injection into a HP-5MS fused silica capillary column (30 m×0.25 mm I.D., HP). The column head pressure was maintained at around 8.2 psi to give a constant flow rate of 1 mL/min using helium as carrier gas. Initial oven temperature was held at 60° C. for 1 min, increased to 140° C. at 25° C./min, to 250° C. at 5° C./min, and then increased to 300° C. at 10° C./min.

The carbohydrate composition analyses of crude Reishi extract indicated that glucose and mannose exist as the major components together with smaller amounts of other sugars, including fucose, N-acetylglucosamine, xylose and rhamnose (Table 1). The crude extract contains 15.6% proteins, the amino acid analysis of which was shown in Table 2.

To further understand the composition and activity of fraction 3, it was treated with protease K to partially destroy the protein component. The result showed that proliferation of Con A-stimulated spleen cells remained the same. Glycolytic cleavage by αI,2-fucosidase, however, abolished the activity of fraction 3 completely (based on MTT assay). In contrast, the activity of fraction 3 was slightly reduced after treatment with αI,3/4-fucosidase. This experiment establishes that the active component is a polysaccharide or glycopeptide containing terminal fucose residues with α1,2-fucosidic linkages. Overall, the main active component is a glycoprotein containing essential terminal fucose residues with α1,2-linkages. The protein moiety is not required for the activity.

Amino acid composition analysis. The analysis was carried out based on a well-established method.⁹ A sample of crude Reishi extract (6 mg) was dissolved in 1 mL solution of 6 M HCl and TFA (4/1), and heated at 140° C. for 3 h. The mixture was concentrated to give a dry residue and dissolved in 100 μL citrate buffer. A small aliquot (4 μL) was withdrawn and subjected to composition analysis by amino acid analyzer (Jeol JLC-6AH).

Sample preparation for proteomic studies. Reishi extract-treated mouse spleen cells were lysed in 350 μL of lysis buffer containing 8 M Urea, 2% CHAPS, 65 mM DTE, 2% v/v isocratic pH gradient (IPG) buffer pH 3-10 NL (non-linear), and a trace of bromophenol blue. The sample was centrifuged for 10 min at 13,000 rpm. The total protein concentration in the sample was measured using Bio-Rad protein concentration assay kit. Samples equal to 500 μg of proteins were loaded on immobilized pH gradient strips (pH 3-10 NL, 18 cm) for 2-dimensional electrophoresis.

2-Dimensional electrophoresis and image processing. The separations were performed as described by Hochstrasser et a1.¹⁰ The isoelectric focusing was carried out in an IPGPhor apparatus (Amersham Pharmacia Biotech). The second dimension was done in 10-15% polyacrylamide gradient gels using thc Protean II xL 2D multi cell (Bio-Rad). Protein spots were stained with fluorescence dye Sypro Ruby™ (Molecular Probes).

Sypro Ruby-stained gels were scanned with fluorescence laser scanner (Bio-Rad) generating 10 Mb image. The images were analyzed with ImageMaster™ software (Amersham Pharmacia Biotech). For each gel the spots were detected and quantified automatically, using default spot detection parameters. Manual spot editing was performed in agreement with the visual inspection of the gels. The relative volume was calculated in order to correct any differences in protein loading and gel staining.

MALDI-TOF MS analysis. Sypro Ruby-stained protein spots were cut from the gel and washed with 200 μL of 50 mM ammonium bicarbonate, pH 8.5, buffer in 50% CH₃CN. Following dehydration in CH₃CN and speed vacuum centrifugation, the gel pieces were swollen in a digestion buffer containing 100 mM ammonium bicarbonate, pH 8.5, 1 mM CaCl₂, 10% CH₃CN and 50 ng of sequencing grade trypsin. The resulting peptides were extracted with 50% CH₃CN/5% TFA after overnight digestion. A 1 μL aliquot of peptide mixture was deposited on the MALDI target 96-well plate and after few seconds 1 μL of a matrix solution (α-cyano-4-hydroxycinnamic acid in 50% CH₃CN/0.1% TFA) was added. The mixture was allowed to dry at ambient temperature. Positive-ion mass spectrum was measured on a MALDI reflection time-of-flight mass spectrometer M@LDI (Micromass UK, Manchester, UK) equipped with a nitrogen laser. The reported spectra were accumulated from 50 to 100 laser shots.

General procedure of fucosidase treatment. A sample of ten mg of Reishi extract or fraction 3 in 50 mM citrate buffer (pH 6.0) was treated with α1,2- or α1,3/4-fucosidase (5 Unit) at 37° C. for a period of time (2-12 h). The mixture was heated in boiling water for 5 min to destroy the enzyme activity, dialyzed against H₂O at 4° C., and lyophilized to give a dry powder for activity studies.

### EXAMPLE 2

Materials. Crude Reishi extract (prepared via alkaline extraction (0.1 N NaOH), neutralization and ethanol precipitation) was obtained from Pharmanex Co. (CA, USA). All the chemicals and reagents were from Sigma Co. (81. Louis, Mo., USA) unless indicated.

Purification of Reishi extract. One hundred grams of crude Reishi extract were dissolved in 3 L of double distilled water, stirred at 4° C. for 24 h, and centrifuged for 1 h to remove the insoluble. The resulting solution was concentrated at 35° C. to give a small volume and lyophilized to generate 70 g powder of dark-brown color, 2.5 g of which were dissolved in a small volume of Tris buffer (pH 7.0, 0.1 N) and purified by gel filtration chromatography using a Sephacryl S-500 column (95 cm×2.6 cm) with 0.1 N Tris buffer (pH 7.0) as the eluent. The flow rate was set at 0.6 mL/min, and 7.5 mL per tube was collected. After the chromatography, each fraction was subjected to anthrone analysis or the phenol-sulfuric acid method to detect sugar components.

Five fractions were collected (fractions 1-5), each dialyzed to remove excessive salt and lyophilized to give 450 mg of fraction 3.

Fraction 3 was further subjected to a column of Diaion-W A30 anion exchanger (Cl-form, 40 cm×3.5 cm) eluted with 0.2 and 0.8 M NaCl at a flow rate of 0.5 mL/min and two fractions were designated as F3G1 (11% yield based on fraction 3) and F3G2 (10% yield based on fraction 3), respectively. Another fraction (F3G3, 11% yield based on fraction 3) was generated when the column was further eluted with 2 M NaOH.

The gel-filtration chromatography of F3G2 was carried out on a TSK HW-75 column (130 cm×2.6 cm) eluted with double distilled water at a flow rate of 0.5 mL/min. There were two fractions collected; i.e., G2H1 (19% yield based on F3G2) and G2H2 (69% yield based on F3G2).

Anthrone colorimetric method.⁸ Each 1.5 mL of anthrone (9,10-dihydro-9-oxoanthracene) solution (0.2 g anthrone dissolved in 100 mL of conc. sulfuric acid) in a series of test tubes immersed in an ice water bath was carefully overlayed with 1.5 mL of sample (20-40 μg/mL of D-glucose or equivalent). After all additions had been made, the tubes were shaken rapidly and then replaced in an ice water bath. The tubes were heated for 5 min a boiling water bath and then cooled; the optical densities were read within an hour at 625 nm against distilled water. Standards, reagent blanks and unknowns were run in triplicate because of likely contamination by other carbohydrate sources. Calculations were made on the basis that the optical densities are directly proportional to the carbohydrate concentration.

Reverse transcription (RT) and polymerase chain reaction (PCR).⁹ Mouse spleen cells were aseptically removed from healthy mice (BALB/c male mice, six weeks old), adjusted to an ideal cell concentration (3×10⁶ cells/mL) and incubated in RPMI-1640 medium containing 10% of FCS (fetal calf serum) at 37° C. with 5% CO2. After 6 h, the cells were subjected to RNA extraction using Qiagen RNAeasy mini kit to obtain ˜I μg of the desired RNA. Reverse transcription (RT) was performed using the Thermoscript R/T PCR System, and the Thermoscript system protocol I, from Gibco BRL. The reaction was carried out as follows: 1 μg of RNA, 1 μL of primer (Oligo(dT)₂O) (SEQ ID NO:23) and 2 μL of 10 mM dNTP Mix were added to each 0.2 mL tube and adjusted the total volume to 12 μL with DEPC H₂O (0.1% diethylpyrocarbonate-treated H₂O). The mixture was incubated at 65° C. for 5 min and immediately chilled on ice. The following was added to each tube as an 8 μL mixture: 4 μL of 5×cDNA buffer, 1 μL of 0.1 M dithiothreitol (DTT), 1 μL of RNaseOut (a ribonuclease inhibitor) and 1 μL of Thermoscript R/T, and 1 μL of DEPC water. The mixture was incubated at room temperature for 10 min and then 50° C. for 1 h to allow first strand of cDNA synthesis. Enzyme activity was terminated by incubating the reactions at 85° C. for 5 min and the tubes were then placed on ice for 10 min. The samples were stored at −20° C. until used for PCR.

Each sample (2 μL) was added to each reaction tube and the following reagents were added as a 25 μL mix: 2.5 μL of 10×PCR buffer, 2 μL of 10 mM dNTP Mix, 2.5 μL of 10 mM each primer (sense and anti-sense), 13 μL of DEPC H₂O, and 1 μL of ProZyme® (DNA polymerase, from PROtech Technology). The reaction tubes were placed in a Strategene PCR Robocycler (Gradient 96) and run under the following condition: 1 cycle at 94° C. for 2 min (initial denaturation), then 25 consecutive cycles of 94° C. for 1 min (denaturation), primer annealing (various temperatures depending on primers, see Table 5 for details) for 1 min and 72° C. for 1 min (primer extension). The reactions were analyzed by gel electrophoresis.

According to the RT-PCR studies for the cytokine expression (Table 4), the treatment with F3G2 led to significant expression of all the ten cytokines aforementioned, which was thus concluded to contain the major active components of fraction 3. The expression of TNF-α and IL-1 were detectable in the studies of F3G1 and F3G3. It is of interest that both fractions can trigger only the inflammatory pathway, unlike fraction 3 or F3G2.

The additional gel-filtration chromatography of F3G2 on a TSK HW-75 column resulted in two fractions—G2H1 (19% yield based on F3G2) and G2H2 (69% yield based on F3G2), as shown in FIG. 6. The preliminary result from the RT-PCR studies revealed that the former fraction contains much higher activity than the same dosage of F3G2 and G2H2 in the expression of IL-1β, IL-6, INF-γ, TNF-α, and GM-CSF.

Sugar composition analysis—TMS method. For monosaccharide analysis, the polysaccharide extracts/fractions were methanolyzed with 0.5 M methanolic-HCl (Supelco) at 80° C. for 16 h, re-N-acetylated with 500 μL of methanol, 10 μL of pyridine and 50 μL of acetic anhydride, and then treated with the Sylon HTP® trimethylsilylating reagent (Supelco) for 20 min at room temperature, dried and redissolved in hexane. GC-MS analysis of the trimethylsilylated derivatives was carried out using a Hewlett-Packard (HP) Gas Chromatograph 6890 connected to a HP 5973 Mass Selective Detector. Samples were dissolved in hexane prior to splitless injection into a HP-5MS fused silica capillary column (30 m×0.25 mm I.D., HP). The column head pressure was maintained at around 8.2 psi to give a constant flow rate of 1 mL/min using helium as carrier gas. Initial oven temperature was held at 60° C. for 1 min, increased to 140° C. at 25° C./min, to 140° C. at 25° C./min, and then increased to 300° C. at 10° C./min.

The carbohydrate composition analyses of crude Reishi extract, fraction 3, F3G1, F3G2 and F3G3 all indicated that glucose and mannose exist as the major components together with smaller components of other sugars including fucose, galactose, N acetylglucosamine and xylose (Table 3). It is of interest that the percentage of galactose decreased significantly in F3G2 and F3G3.

MNC Isolation

To isolate mononuclear cells (MNCs), each umbilical cord blood (CB) or human peripheral blood (PB) unit was diluted 1:1 with phosphate-buffered saline (PBS)/2 mM EDTA, and carefully loaded onto Ficoll-Hypaque solution. After density gradient centrifugation at 2000 rpm for 40 minutes at room temperature, MNCs were removed from the interphase and washed two to three times with PBS/EDTA.

Dendritic Cell (DC) Culture

2×10⁷ PBMNCs were plated on 25 cm² tissue culture flasks (Cloning) in 4 ml RPMI (Invitrogen) medium after 1 h incubation at 37° C. in humidified atmosphere containing 5% carbon dioxide, nonadherent cells were removed and adherent cells were cultured in serum-free AIM-V medium supplemented with human cytokine IL-4 and GM-CSF (R&D) with/without F3. During culture 7 days period, PBMNCs were transferred to dendritic cell morphology.

HSC Ex-Vivo Expansion in a Co-Culture System

The CD34+ cells were enriched using a magnetic activated cell sorting (MACS) CD34 isolation kit. Usually 2-5×10⁵ CD34+ cells could be obtained from 1×10⁸ MNCs with 90-95% purity, as confirmed by FACS analysis. Murine stomal cell line (MS-5) was used as a stromal layer for the co-culture of CD34+ cells. MS-5 was cultured more than one week in low-glucose DMEM supplemented with 10% FBS, and PSN antibiotics. The purified CD34+ cells density of 1×10⁴ cells/mL were resuspended in 4 mL serum-free EX-VIVO 10 medium supplemented with 2 mmol/L 1-glutamine, PSN antibiotics, 10 U/ml recombinant human thrombopoietin (rhTPO; R&D), 50 ng/mL stem cell factor (SCF; R&D), 50 ng/mL flt3-ligand (FL; R&D systems, Minneapolis, Minn., USA), and 10 ng/mL interleukin-6 (Il-6; R&D systems, Minneapolis, Minn., USA). The purified CD34+ cells co-culture with MS-5 feeder in 25 T flask. At day 8, the suspended cells were harvested after the plates had been gently shaken. The remaining weakly attached hematopoietic cells were completely recovered by adding 3 mL DMEM to each well. The total cell and viable cell numbers were estimated using a hematocytometer by counting the number of trypan blue unstained cells under an optical microscope.

AD-MSCs Isolation and Chondrogenesis

AD-MSCs were obtained from raw human lipoaspirates and cultured as described in Chien et. al., Bioorganic & Medicinal Chemistry 12, 5603-5609 (2004), herein expressly incorporated by reference. Briefly, raw lipoaspirates were washed extensively with sterile phosphate-buffered saline (PBS) to remove contaminating debris and red blood cells. Washed aspirates were treated with 0.075% collagenase (type I; Sigma-Aldrich, St. Louis, Mo.) in PBS for 30 min at 37° C. with gentle agitation. The collagenase was inactivated with an equal volume of DMEM/10% fetal bovine serum (FBS) and the infranatant centrifuged for 10 min at low speed. The cellular pellet was resuspended in DMEM/10% FBS and filtered through a 100-μm mesh filter to remove debris. The filtrate was centrifuged as detailed above and plated onto conventional tissue culture plates in maintain medium. AD-MSCs were maintain in DMEM-LG (GIBCO) supplemented with 10% FBS (Hyclone)AD-MSCs were maintain in DMEM-LG (GIBCO) supplemented with 10% FBS (Hyclone). In vitro differentiations of MSCs were treated with chondrogenic medium: DMEM-LG supplemented with 1% FBS, 6.25 μg/ml insulin, 10 ng/ml TGF-91 (R&D), and 50 nM ascorbate-2-phosphate. For chondrogenic differentiation, a higher cell density of 1 to 2×10⁵ per 10 μl was used for chondrosphere formation. During culture chondrocytes were counted at 3 days and at 7 days.

RT-PCR

Total cellular RNA was isolated using RNeasy total RNA isolation kit (Qiagen), and cDNA was reversed using the SuperScript First-strand Synthesis System (Invitrogen). Specific genes were amplified by PCR using Fast-Run Taq Master Kit (Protech Technology, Taipei, Taiwan).

FACS Analysis

Adult peripheral blood derived dendritic cells and cord blood expanded HSC were characterized by FACS for specific surface antigens. Harvested cells were collected and stained with fluorescein isothiocyanate- or phycoerythrin-conjugated anti-marker monoclonal antibodies in 100 μl phosphate buffer using titers for 15 minutes at room temperature, as suggested by the manufacturer. Cell surface markers included dendritic cells markers (CD1a, CD40, CD80, CD83, CD86) and hematopoietic lineage markers (CD34, CD38, CD133, CXCR4). Cells were analyzed using a flow cytometry system (FACSCalibur; Becton, Dickinson). Positive cells were counted and compared with the signal of unstained cells.

Human Cytokine Array Analysis

Collected PBMNCs were transferred into dendritic cells during 7 days conditional medium. The culture medium is AIM-V supplemented with human cytokine IL-4 and GM-CSF (R&D) with or without F3. Cell—secreted cytokines were detected by RayBiot™ human cytokine antibody array kit (RayBiotech, Norcross, Ga.). The cytokine antibody array membrane was blocked 30 mins at room temperature. The blocking solution was removed then 2 ml conditional medium incubated with membrane 1.5 hr. The membrane were washed with wash buffer I, II then biotin-conjugated antibody was added to membrane for 1 hr. After washing, HRP-conjugated streptavidin was added to membrane for 40 mins, then X-ray film exposed by ACL reagent. The image used KODAK 1D 3.5 software to analyze, then normalize data to draw a graph by EXCEL.

Results:

F3 influences cell adhesion molecule (CAM) expressions in hematopoietic mononuclear cells (MNCs):

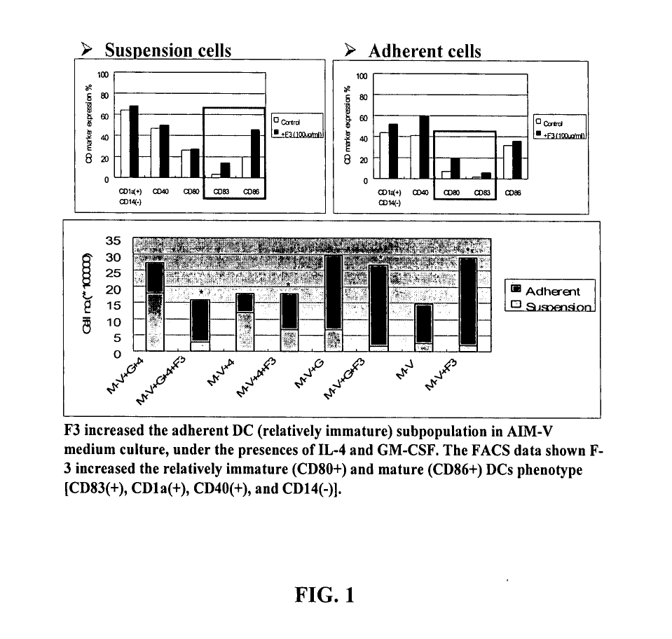

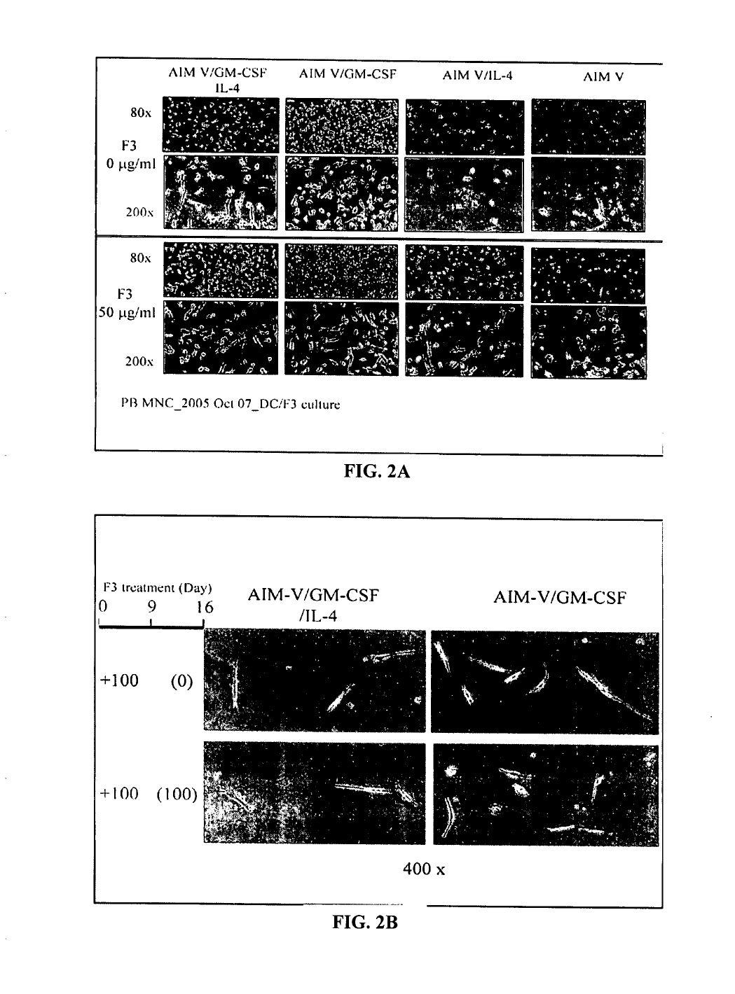

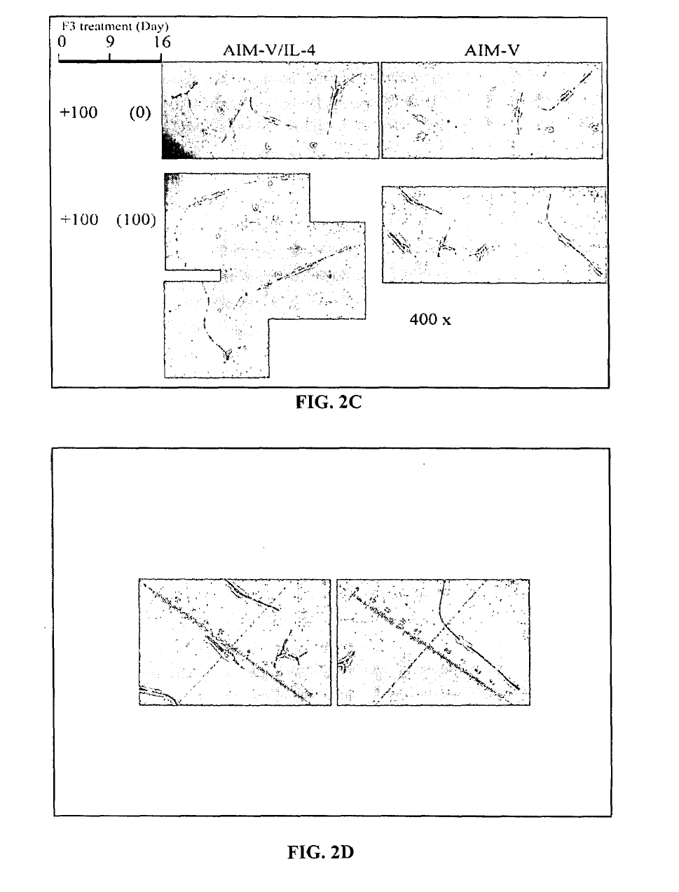

Cell adhesion molecules (CAM) and co-stimulatory factors of adherent mononuclear cells are known to be important in the activation of eukaryotic immune system cells and anti-cancer metastasis. In this study, F3 significantly increased the CB-MNC attachment in the culture dish, and induced cellular morphological changes, regardless of the presence of cytokines (GM-CSF (G), IL-4 (4)) under AIM-V (M-V) basal culture media (FIGS. 1, 2A-2E).

F3 Enhances Immature Dendritic Cells (Daces) Formation and B Cell Production:

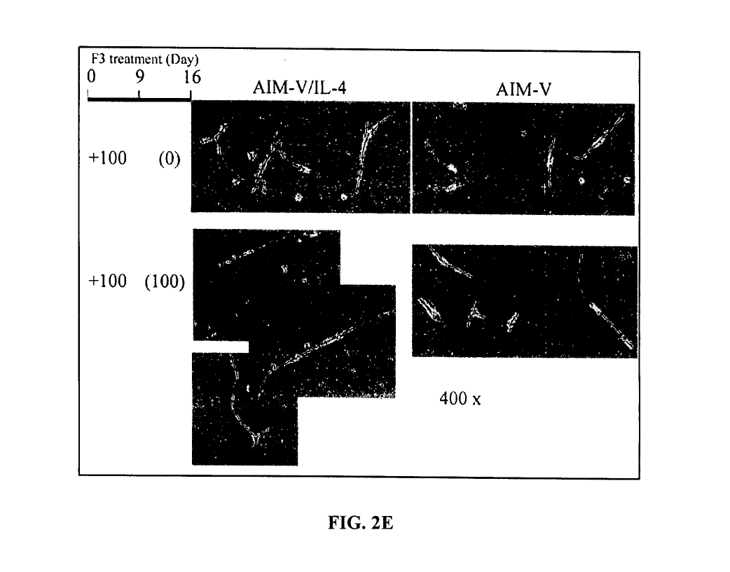

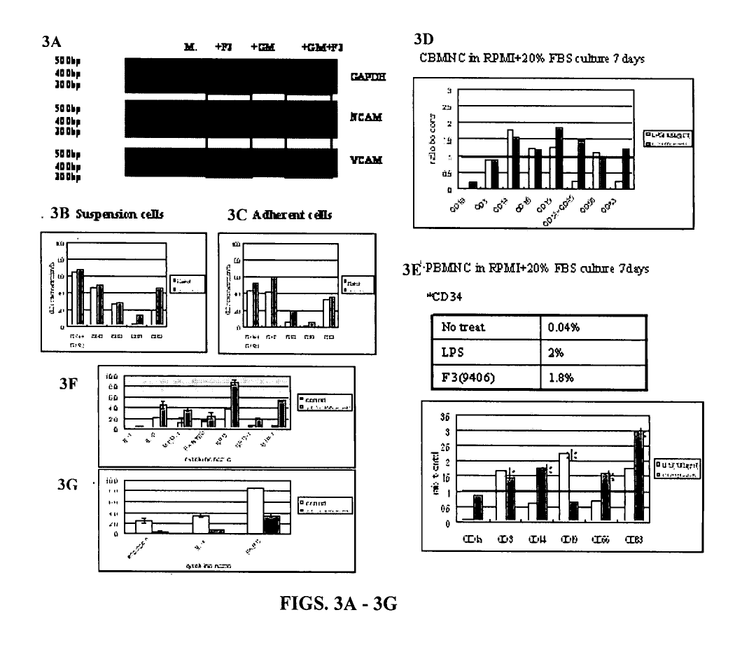

Dendritic cells, the mononuclear cells that initiate immune response, and Granulocyte-macrophage colony-stimulating factor (GM-CSF) and macrophage colony-stimulating factor (M-CSF) together with IL-4 are known to reciprocally regulate the MNC to DC trans-differentiation. Upon examining the influence of F3 on the CB-MNC to DC transformation, we found F3 specifically enhances I super family CAM (N-CAM and V-CAM) expressions (FIG. 3A) but not the I-CAM nor PE-CAM. Adhesive CB-MNCs stimulated by F3 not only resulted in making more CD83+ mature dendrite cell (DC) in RPMI basal medium (FIG. 3D-E), as previously described by C. M. Chien et al. (herein expressly incorporated by reference), but also may produce more active immature adherent DC (CD1a+, CD40+, CD80+, and CD86+) subset, with enhanced the cell dendrite growing under the AIM-V medium culture condition (FIGS. 3B-3C). CB- and PB-MNCs respond differently to the F3 stimulation in transforming into immature DC cells.

F3 may serve as an effective adjuvant for transforming monocytes to generate more immature and active DCs for cancer immunotherapy. Furthermore, upon the stimulation of Reishi F3, we also found that the CD19+ subpopulation B cells were significantly increased in CB-MNCs to DC culture (FIG. 3D).

Immature DC-OC trans-differentiation has been shown greatly enhanced by rheumatoid arthritis synovial fluid and involves proinflammatory cytokines such as IL-1 or TNF-a, as well as components of the ECM such as hyaluronic acid (Immature dendritic cell transdifferentiation into osteoclasts: a novel pathway sustained by the rheumatoid arthritis microenvironment Blood. 2004; 104:4029-4037, hereby expressly incorporated by reference). F3 influences the up regulations of pro-inflammatory cytokines and down regulations of the PARK and angiogenin expressions of adult blood, in a dendritic cell culture, under co-stimulations of GM-CSF and IL-4 for 7 days. IL-1β, IL-6, MCP-1, RANTES, GRO, GRO-α, MIP-β were up regulated, as contrast to the down regulated PARC, and angiogenin cytokines analyzed by a human cytokine array (FIGS. 3F-3G).

F3 may serve as an effective adjuvant for transforming CB- or PB-MNC into adherent immature active DCs (FIG. 1), and may influence the CD19+B cell populations in the cell culture (FIG. 3D).

F3 Maintenance of the primitive hematopoietic CD34+ stem cell population in vitro Expansion of CB-HSCs:

Sovalat H, et al., J. Hematother Stem Cell Res. 12, 473-489 (2003)(herein expressly incorporated by reference) demonstrated that decreasing adhesion of HSCs to BM stromal cells in mobilized blood was due to a down regulation of adhesion molecules of the CD34+ cells and CD34+CD38− proteome subset, in compared to those from steady-state blood, bone marrow, and cord blood.

In this study, CD34+ HSCs from CB were isolated and subjected to examine the F3 influences of CD34+ HSCs survival and proliferation by a five cytokine cocktail liquid culture and a stromal cell based co-culture system. As shown in FIGS. 4A-4C, Reishi extract and particularly the F3 fraction of Reishi extract retains CD38− and CD133+ primitive HSC subpopulations of CD34+ HS/PCs in both liquid, and feeder co-culture systems, indicating that F3 may serve as a soluble matrix for preventing the primitive CD38− and CD133+ subpopulations of CD34+ HSCs from differentiation in the ex-vivo expansion culture.

By examining the F3 hydrolyzed components we have further identified Reishi extract fractions that enhance the CD38− and CD133+ primitive (FIGS. 4D-4E) and CXCR4+ HSC CD34+ (FIG. 4F) populations. Our data indicated that the glycoside moiety loss in liquid culture may be prevented by supplementing selective natural glycosides and/or glycoproteins, such as F3, as soluble scalford for maintaining primitive HSCs in undifferentiated state for the ex-vivo expansion culture. Therefore selective glycosides or glycoproteins, such as F3, may be supplemented in ex-vivo expansion co-culture of CB-HSCs and treating the leukaphresis treated mobilized PB-HSCs for better transplant success.

F3 Influences the MSC Chondrogenic Differentiation Potentials

N-CAM has been shown critical in CNS development, involved in neurite out-growth, axon guidance, and migration, and activate signal receptor induce intracellular signal cascades. Fang, J. H. B. Int J. Dev. Bio. 43:335-342 (1999) (hereby expressly incorporated by reference). N-CAM expression has also been shown involved in skeletal condensation and initiating chondrogenesis (mediating the formation of precartilagious condensation) in the early chondrogenesis (Widelitz et. al., J. Cell. Physiol. 156: 399-411 (1993) (hereby expressly incorporated by reference).

Upon examination of F3 influence in a chondrosphere induction mesenchymal stem cell differentiation culture, it was found that F3 accellarates and enhances mesenchymal chondrosphere formation, accompanied by increased BMP-2, IL-1, and aggrecan gene expressions (FIGS. 5A-5C). Based on these results F3 may be useful for skeleton remodeling drug by co-formulation with skeleton forming agents.

TABLE 1
Carbohydrate compositions of crude Reishi extract.

| Sugar components | Percentage (%) |
|---|---|
| D-glucose | 58.0 |
| D-mannose | 15.5 |
| L-fucose | 9.7 |
| D-galactose | 9.3 |
| D-xylose | 5.4 |
| D-GlcNAc | 1.0 |
| L-Rham | 0.5 |

TABLE 2
Amino acid analysis of Reishi extract.

| | Relative | | Relative |
|---|---|---|---|
| Amino acid | abundance | Amino acid | abundance |
| Asp | 117 | Met | 6 |
| Thr | 66 | Ile | 36 |
| Ser | 54 | Leu | 55 |
| Glu | 120 | Tyr | 16 |
| Pro | 60 | Phe | 28 |
| Gly | 108 | His | 12 |
| Ala | 100 | Lys | 21 |
| Val | 61 | Arg | 22 |

TABLE 3
Carbohydrate compositions of fraction 3, F3G1, F3G2, and F3G3

| | Percentage (%) | | | | | |
|---|---|---|---|---|---|---|
| | L-Fucose | D-Xylose | D-Mannose | D-Galactose | D-GlcNAc | D-Glucose |
| fraction 3 | 7.1 | 3.1 | 15.1 | 13.5 | 1.20 | 58.1 |
| F3G1 | 8.0 | 5.7 | 10.2 | 12.6 | 0.25 | 63.2 |
| F3G2 | 6.2 | 4.5 | 18.3 | 5.3 | 0.78 | 64.9 |
| F3G3 | 8.4 | 7.2 | 14.5 | 2.9 | 1.18 | 65.7 |

TABLE 4
Cytokine expression of mouse splenocytes treated with different Reishi samples^{a}

| | | cytokine expression^{b} | | | | | | | | | |
|---|---|---|---|---|---|---|---|---|---|---|---|
| entry | samples | IL-1β | IL-2 | IL-4 | IL-6 | INF-γ | IL-12 | TNF-α | GM-CSF | G-CSF | M-CSF |
| 1 | crude Reishi extract | + | − | − | ± | ± | + | + | − | + | + |
| 2 | fraction 3 | + | − | − | + | + | + | + | + | + | + |
| 3 | F3G1 | + | − | − | − | − | + | + | − | + | ± |
| 4 | F3G2 | + | − | − | + | + | + | + | + | + | + |
| 5 | F3G3 | + | − | − | − | − | ± | + | − | ± | ± |

^{a}Each sample was evaluated at four concentrations including 10², 10¹, 10⁰ and 10⁻¹ μg/mL. The cells (3 × 10⁶ cells/mL) were incubated at 37° C. with 5% CO₂.
^{b}+, indicating a significant increase of cytokine expression; −, indicating no increase of cytokine expression; ±, showing an increase but not significant of cytokine mRNAs.

TABLE 5
Primer Sequences Used in RT-PCR Experiments

| | | | | |
|---|---|---|---|---|
| | | | | Size of |
| | | SEQ | Annealing | PCR |
| | | ID | temp | product |
| Cytokine | Sequences (S: sense, A: anti-sense) | NO | (° C.) | (bp) |

| | | | | |
|---|---|---|---|---|
| IL-1β | S 5′-CAACCAACAAGTGATATTCTCCATG-3′ | 1 | 55 | 152 |
| | A 5′-GATCCACACTCTCCAGCTGCA-3′ | 2 | | |
| IL-2 | S 5′-TGATGGACCTACAGGAGCTCCTGAG-3′ | 3 | 59 | 167 |
| | A 5′-GAGTCAAATCCAGAACATGCCGCAG-3′ | 4 | | |
| IL-4 | S 5′-ACGAGGTCACAGGAGAAGGGACGCCATGCA-3′ | 5 | 71 | 221 |
| | A 5′-TCATTCATGGAGCAGCTTATCGATGAATCC-3′ | 6 | | |
| IL-6 | S 5′-GTGACAACCACGGCCTTCCCTACT-3′ | 7 | 53 | 313 |
| | A 5′-GGTAGCTATGGTACTCCA-3′ | 8 | | |
| IL-12 | S 5′-TGTTGTAGAGGTGGACTGG-3′ | 9 | 67-65 | 483 |
| | A 5′-TGGCAGGACACTGAATACTT-3′ | 10 | | |
| IFN-γ | S 5′-TGGAGGAACTGGCAAAAGGATGGT-3′ | 11 | 63 | 336 |
| | A 5′-TTGGGACAATCTCTTCCCCAC-3′ | 12 | | |
| TNF-α | S 5′-GCGACGTGGAACTGGCAGAAG-3′ | 13 | 65 | 383 |

1. A method comprising:
administering a sufficient amount of purified reishi extract to a subject;
so that the mononuclear cell expression of cell surface markers is increased by at least 1%.

2. The method of claim 1, wherein the cell surface markers are VCAM and/or NCAM.

3. The method of claim 1, wherein the cell surface markers are selected from the group consisting of immature dendritic cell markers.

4. The method of claim 3, where the immature dendritic cell markers are selected from the group consisting of CD1a, CD14, CD40, CD80, and CD86.

5. The method of claim 1, wherein the cell surface markers are selected from the group consisting of mature dendritic cell markers.

6. The method of claim 5, wherein the mature dendritic cell markers are selected from the group consisting of CD83.

7. The method of claim 1, wherein the cell surface markers are selected from the group consisting of hematopoietic cell markers.

8. The method of claim 7, wherein the hematopoeitic cell markers are selected from the group consisting of CD34, CD38, CD133, and CXCR4.

9. The method of claim 1, wherein the cell surface markers are selected from the group consisting of B cell markers.

10. The method of claim 9, wherein the B cell markers are selected from the group consisting of CD19.

11. The method of any of claim 3, wherein purified reishi is co-administered to a subject with at least one cytokine selected from the group consisting of IL-4 and GM-CSF.

12. A method comprising:
administering a sufficient amount of purified reishi to a subject;
so that the MSC and/or PLA expression of cell surface markers is increased by at least 1%.

13. The method of claim 12, wherein the cell surface markers are selected from the group consisting of BMP-2, aggrecan, and IL-1.

14. The method of claim 12, wherein purified reishi is co-administered to a subject with at least one compound selected from the group consisting of insulin, TGF-B1, and/or ascorbate-2-phosphate.

15. A method comprising:
administering a sufficient amount of purified reishi to a subject; so that the percentage of subject MSC and/or PSA cells that lose expression of the CD34+/CD38− cell proteome is decreased by at least 1%.

16. A device comprising:
an amount of skeleton forming agent;
an amount of reishi extract; and
wherein the device may be implanted

17. The device of claim 16, further comprising an amount of mononuclear cells.

18. The device of claim 16, further comprising an amount of MSC and/or PLA


SEQUENCE LISTING (13 sequences)

SEQ ID NO: 1 - DNA, 25 bp, Artificial Sequence
  Primer
caaccaacaagtgatattctccatg

SEQ ID NO: 2 - DNA, 21 bp, Artificial Sequence
  Primer
gatccacactctccagctgca

SEQ ID NO: 3 - DNA, 25 bp, Artificial Sequence
  Artificial Sequence
tgatggacctacaggagctcctgag

SEQ ID NO: 4 - DNA, 25 bp, Artificial Sequence
  Primer
gagtcaaatccagaacatgccgcag

SEQ ID NO: 5 - DNA, 30 bp, Artificial Sequence
  Primer
acgaggtcacaggagaagggacgccatgca

SEQ ID NO: 6 - DNA, 30 bp, Artificial Sequence
  Primer
tcattcatggagcagcttatcgatgaatcc

SEQ ID NO: 7 - DNA, 24 bp, Artificial Sequence
  Primer
gtgacaaccacggccttccctact

SEQ ID NO: 8 - DNA, 18 bp, Artificial Sequence
  Primer
ggtagctatggtactcca

SEQ ID NO: 9 - DNA, 19 bp, Artificial Sequence
  Primer
tgttgtagaggtggactgg

SEQ ID NO: 10 - DNA, 20 bp, Artificial Sequence
  Primer
tggcaggacactgaatactt

SEQ ID NO: 11 - DNA, 24 bp, Artificial Sequence
  Primer
tggaggaactggcaaaaggatggt

SEQ ID NO: 12 - DNA, 21 bp, Artificial Sequence
  Primer
ttgggacaatctcttccccac

SEQ ID NO: 13 - DNA, 21 bp, Artificial Sequence
  Primer
gcgacgtggaactggcagaag